(19) United States
(12) Patent Application Publication
(10) Pub. No.: US 2022/0031796 A1
(43) Pub. Date: Feb. 3, 2022

(54) LIQUID COMPOSITION FOR ORAL USE AND METHOD FOR REDUCING BITTERNESS OF LIQUID COMPOSITION FOR ORAL USE

(71) Applicant: SUNTORY HOLDINGS LIMITED, Osaka-shi, Osaka (JP)

(72) Inventors: Wakana Taguchi, Soraku-gun (JP); Takako Imao, Soraku-gun (JP)

(73) Assignee: SUNTORY HOLDINGS LIMITED, Osaka-shi, Osaka (JP)

(21) Appl. No.: 17/297,759

(22) PCT Filed: Nov. 29, 2019
(86) PCT No.: PCT/JP2019/046713

§ 371 Date: May 27, 2021

(30) Foreign Application Priority Data
Nov. 30, 2018 (JP) 2018-226088

(51) Int. Cl. A61K 38/05; A61K 47/10; A23L 2/52; A23L 33/18; A23L 27/00

(52) U.S. Cl. CPC A61K 38/05; A61K 47/10; A23V 2002/00; A23L 33/18; A23L 27/86; A23L 2/52

(57) ABSTRACT

The present invention aims to provide a technique that reduces the bitterness of Hyp-Gly in liquid oral compositions containing a specific amount of Hyp-Gly. The present invention relates to, but is not limited to, a liquid oral composition containing Hyp-Gly in an amount of 1.0 mg/100 mL or more and a C2-C4 mono- to trihydric alcohol.

### TECHNICAL FIELD

The present invention relates to a liquid oral composition. The present invention also relates to, for example, a method of reducing the bitterness of a liquid oral composition.

### BACKGROUND ART

Collagen is a protein that has been widely used as gelatin in the food field. Generally, it is believed hard to use a high molecular weight collagen efficiently in the body when it is orally ingested. Recently, collagen peptides, which are low molecular weight collagen molecules broken down from high molecular weight collagen molecules, have been developed to be suitable for ingestion in the body.

Various functions of collagen peptides have been discovered. For example, Hyp-Gly (OG), which is a dipeptide containing hydroxyproline (Hyp) derived from collagen, is reported to have an effect that is good on the skin and the like.

Continuous intake of collagen peptides is important in order to benefit from the effects of collagen peptides such as Hyp-Gly. Yet, many types of collagen peptides have unique odor and taste bitter. Thus, studies have been made on methods of improving the flavor. Patent Literature 1 discloses a method of masking unpleasant odor and/or bitterness derived from collagen peptides in a soft drink containing collagen peptides. This method uses a milk solid component containing a non-fat milk solid component in an amount that is effective for masking the unpleasant odor and/or bitterness.

### CITATION LIST

### Patent Literature

Patent Literature 1: JP 2014-117254 A

### SUMMARY OF INVENTION

### Technical Problem

As described above, Hyp-Gly (hydroxyprolyl-glycine, hereinafter also referred to as “OG”) has been reported to have some useful effects. However, for example, a beverage containing Hyp-Gly in an amount of 1.0 mg/100 mL or more tastes bitter from Hyp-Gly, which is a factor that makes continuous oral intake difficult. This indicates usefulness of a technique that can reduce the bitterness of Hyp-Gly in a liquid oral composition, such as beverage, containing a specific amount of Hyp-Gly. Patent Literature 1 discloses a method of masking the bitterness and the like derived from collagen peptides, but is silent about studies on methods of reducing the bitterness of a liquid oral composition containing a specific amount of Hyp-Gly.

The present invention aims to provide a technique that reduces the bitterness of Hyp-Gly in a liquid oral composition containing a specific amount of Hyp-Gly.

### Solution to Problem

As a result of extensive studies to solve the above problem, the present inventors found that adding a C2-C4 mono- to trihydric alcohol to a liquid oral composition containing Hyp-Gly in an amount of 1.0 mg/100 mL or more can reduce (suppress) the bitterness of Hyp-Gly.

Specifically, the present invention relates to a liquid oral composition, a method of reducing the bitterness of a liquid oral composition, and the like described below.

(1) A liquid oral composition containing: Hyp-Gly in an amount of 1.0 mg/100 mL or more; and a C2-C4 mono- to trihydric alcohol.

(2) The liquid oral composition according to (1) above, wherein an amount of the alcohol is 0.001 to 1.0 vol %.

(3) The liquid oral composition according to (1) or (2) above, wherein the alcohol is at least one selected from the group consisting of ethanol, butylene glycol, propylene glycol, and glycerol.

(4) The liquid oral composition according to any one of (1) to (3) above, wherein an amount of the Hyp-Gly is 1.0 to 100 mg/100 mL.

(5) The liquid oral composition according to any one of (1) to (4) above, wherein the liquid oral composition is a beverage.

(6) A method of reducing the bitterness of a liquid oral composition, the method including: adding a C2-C4 mono- to trihydric alcohol to a liquid oral composition containing Hyp-Gly in an amount of 1.0 mg/100 mL or more.

### Advantageous Effects of Invention

The present invention can reduce the bitterness of Hyp-Gly in a liquid oral composition, such as beverage, containing a specific amount of Hyp-Gly.

### DESCRIPTION OF EMBODIMENTS

<Liquid Oral Composition>

The liquid oral composition of the present invention contains Hyp-Gly in an amount of 1.0 mg/100 mL or more and a C2-C4 mono- to trihydric alcohol. In the present invention, Hyp-Gly (OG) refers to a peptide (dipeptide) consisting of an amino acid sequence represented by hydroxyproline-glycine. In the present invention, preferably, Hyp-Gly is a linear dipeptide. Herein, the C2-C4 mono- to trihydric alcohol is sometimes simply referred to as the “alcohol”.

Herein, a peptide is described according to a conventional method such that the N-terminus (amino terminus) is on the left side and the C-terminus (carboxy terminus) is on the right side.

In the present invention, the bitterness derived from Hyp-Gly in a liquid oral composition containing Hyp-Gly in an amount of 1.0 mg/100 mL or more can be reduced by adding a C2-C4 mono- to trihydric alcohol to the liquid oral composition. The amount of Hyp-Gly in the liquid oral composition may be more than 1.4 mg/100 mL. The amount is preferably 1.5 mg/100 mL or more, more preferably 25 mg/100 mL or more, still more preferably 28 mg/100 mL or more, and is also preferably 100 mg/100 mL or less, more preferably 35 mg/100 mL or less. Herein, the range may be any combination of any upper limit and any lower limit. In one embodiment, in order to reduce the bitterness of Hyp-Gly, the amount of Hyp-Gly in the liquid oral composition is preferably 1.0 to 100 mg/100 mL, more preferably 1.0 to 35 mg/100 mL, still more preferably more than 1.4 mg/100 mL to 35 mg/100 mL or less, yet still more preferably 1.5 to 35 mg/100 mL, yet still even more preferably 25 to 35 mg/100 mL, particularly preferably 28 to 35 mg/100 mL.

The source and production method of Hyp-Gly are not limited. Hyp-Gly prepared by a method known in the relevant field can be used. Collagen peptides containing Hyp-Gly can be obtained, for example, by hydrolysis of collagen or modified collagen such as gelatin. The liquid oral composition may be prepared using such collagen peptides containing Hyp-Gly obtained as described above. Hyp-Gly can be prepared by suitably purifying collagen peptides containing Hyp-Gly which are obtainable by hydrolysis of collagen or modified collagen. Hyp-Gly can also be produced by chemical synthesis.

In the liquid oral composition of the present invention, collagen peptides containing Hyp-Gly may be added to the liquid oral composition such that the amount of Hyp-Gly in the liquid oral composition is 1.0 mg/100 mL or more. Collagen peptides containing Hyp-Gly may contain only Hyp-Gly or may contain another peptide in addition to Hyp-Gly.

Collagen peptides containing Hyp-Gly can be obtained by hydrolysis of collagen or modified collagen such as gelatin with an enzyme, acid, alkali, or the like. The source and production method of collagen peptides are not limited. Artificially synthesized collagen peptides can also be used. One type of collagen peptides may be used alone or two or more types of collagen peptides may be used in combination.

Collagen or gelatin as a raw material of the collagen peptides may be one from bovine, swine, chicken, fish, or the like. One or more of these can be used as raw materials. In one embodiment, collagen from fish is preferred. Fish may be saltwater fish or freshwater fish. Examples include tuna (yellowfin), shark, cod, olive flounder, righteye flounder, sea bream, tilapia, salmon, and catfish.

Any enzyme may be used to prepare the collagen peptides as long as the enzyme can cleave peptide bonds of collagen or gelatin. Examples include collagenase, papain, bromelain, actinidine, ficin, cathepsin, pepsin, chymosin, trypsin, and enzymatic preparations in which these enzymes are mixed. The acid may be, for example, hydrochloric acid, sulfuric acid, nitric acid, or the like. The alkali may be, for example, sodium hydroxide, calcium hydroxide, or the like.

In the present invention, an aqueous solution of hydrolyzed collagen peptides may be used as it is or a hydrolyzed collagen peptide powder obtained by drying or the like may be used. Alternatively, the aqueous solution subjected to a usual purification treatment may be used in the form of an aqueous solution, a powder, or the like.

Commercially available collagen peptides may be used. When the amount of Hyp-Gly in collagen peptides is less than a specific amount, for example, Hyp-Gly may be suitably added.

When collagen peptides containing Hyp-Gly are used, the average molecular weight of the collagen peptides is not limited, and for example, it is preferably 300 to 5000, more preferably 300 to 4000.

Herein, the average molecular weight of the collagen peptides is the weight average molecular weight. Herein, the average molecular weight of the collagen peptides means the value measured by a relative molecular mass measurement method in Chinese National Standards (GB standards) GB/T 22729-2008: oligopeptides powder of marine fish. Yet, substitution products are used as reagents for M, 451 and M, 189.

In this method, substances whose molecular weights are known such as cellular pigment C (cytochrome, M, 6500), Trasylol (aprotinin, M, 12500), Bacillus (bacitracin, M, 1450), glycine-glycine-tyrosine-arginine (M, 451), and glycine-glycine-glycine (M, 189) are measured in advance under the same conditions to obtain a relative molecular mass calibration curve showing a relationship between retention time and logarithm of relative molecular weight. The average molecular weight of the collagen peptides is calculated based on the calibration curve. The average molecular weight herein means the weight average molecular weight calculated in terms of each standard substance according to this method.

The amount of Hyp-Gly in the liquid oral composition or in the collagen peptides can be measured using LC/MS/MS, for example, by the following method. For example, an LC/MS/MS device may be LCMS-8050 available from Shimadzu Corporation. For example, a pump may be LC-30AD or the like available from Shimadzu Corporation. For example, a column oven may be CTO-20AC or the like available from Shimadzu Corporation. For example, a 1% aqueous solution of formic acid can be used for dilution of a sample or the like.

(LC Analysis Conditions)

Column: Intrada Amino Acid (Prod No. WAA34, Ser No. PEO9HQF available from Imtakt), 3 μm, 3.0 mm I.D.×100 mm

Column temperature: 35° C.

Flow rate: 0.6 mL/min

Eluent A: acetonitrile with 0.1% formic acid

Eluent B: 100 mM aqueous solution of ammonium formate

Gradient: Eluent B (vol %) 14% (0 min)—14% (6 min)—100% (20 min)—14% (20.1 min)—14% (24 min)

LC end time: 24 min

Amount of injection: 1 μL

Initial pressure after equilibrium: pump A: 6.3 MPa; pump

B: 6.4 MPa

(MS Analysis Conditions)

Ionization mode: ESI Positive

Nebulizer gas flow rate: 3 L/min

Drying gas flow rate: 10 L/min

DL temperature: 250° C.

Block heater temperature: 400° C.

Interface temperature: 300° C.

Heating gas flow rate: 10 L/min

Analysis Mode:

MRM (+) 189.05>86.10

Q1 Pre Bias (V): −22.0, CE: −16.0,

Q3 Pre Bias (V): −13.0

The C2-C4 mono- to trihydric alcohol used in the present invention is preferably an alcohol usable in foods and beverages. Examples thereof include ethanol, propanol, isopropanol, butylene glycol, propylene glycol, and glycerol. Only one alcohol may be used, or two or more alcohols may be used in combination. In particular, in order to reduce the bitterness, at least one selected from the group consisting of ethanol, butylene glycol, propylene glycol, and glycerol is preferred; ethanol and/or propylene glycol are/is more preferred; and ethanol is still more preferred.

In order to reduce the bitterness, the amount of the C2-C4 mono- to trihydric alcohol in the liquid oral composition of the present invention is preferably 0.001 vol % (v/v %) or more, more preferably 0.005 vols or more, still more preferably 0.01 vol % or more. The amount of the alcohol in the liquid oral composition is preferably 1.0 vol % or less. Even when the amount of the alcohol is more than 1.0 vol %, the effect of reducing the bitterness may not be enhanced. In order to more sufficiently obtain the effect of reducing the bitterness, the amount of the alcohol in the liquid oral composition is preferably less than 1.0 vol %, more preferably 0.5 vol % or less, still more preferably 0.3 vol % or less, particularly preferably 0.1 vol % or less. In order to reduce the bitterness, the amount of the alcohol in the liquid oral composition of the present invention is preferably 0.001 to 1.0 vol %, more preferably 0.001 vol % or more and less than 1.0 vol %, still more preferably 0.005 to 0.5 vol %, yet still more preferably 0.005 to 0.3 vol %, particularly preferably 0.01 to 0.3 vol %, most preferably 0.01 vol % to 0.1 vol %. The amount of the C2-C4 mono- to trihydric alcohol is the total amount of two or more alcohols when the liquid oral composition contains two or more alcohols.

The amount of the alcohol can be measured by a known method. The amount of ethanol can be measured by gas chromatography, for example. The amount of propylene glycol can be measured by gas chromatography-mass spectrometry, for example.

In one embodiment, the ratio of the amount of the C2-C4 mono- to trihydric alcohol (vol %) to the amount of Hyp-Gly (mg/100 mL) in the liquid oral composition (i.e., the amount of alcohol (vol %)/the amount of Hyp-Gly (mg/100 mL)) is, for example, preferably 0.00003 or more, more preferably 0.00025 or more, more preferably 0.0007 or more, and is also preferably 0.7 or less, more preferably 0.5 or less, still more preferably 0.07 or less, yet still more preferably 0.05 or less, particularly preferably 0.04 or less, most preferably less than 0.04.

In one embodiment, the ratio of the amount of the C2-C4 mono- to trihydric alcohol (vol %) to the amount of Hyp-Gly (mg/100 mL) in the liquid oral composition (i.e., the amount of alcohol (vol %)/the amount of Hyp-Gly (mg/100 mL)) is preferably 0.00003 to 0.7, more preferably 0.00025 to 0.5, still more preferably 0.00025 to 0.07, yet still more preferably 0.00025 to 0.05, particularly preferably 0.0007 to 0.05. In one embodiment, the ratio of the amount of the C2-C4 mono- to trihydric alcohol (vol %) to the amount of Hyp-Gly (mg/100 mL) is preferably 0.00025 to 0.04, more preferably 0.00025 or more and less than 0.04.

The liquid oral composition of the present invention may contain one or more components other than the above-described components as long as the effect of the present invention is not impaired.

The liquid oral composition of the present invention may contain, for examples, one or more additives. Examples thereof include sweeteners (e.g., erythritol, acesulfame K, and sucralose), acidulants (e.g., citric acid), antioxidants, stabilizers, preservatives, flavoring or masking agents, emulsifiers, pigments, seasonings, pH adjusters, nutritional enhancers, and thickening agents (e.g., welan gum and xanthan gum). In addition to Hyp-Gly, the liquid oral composition of the present invention may also contain one or more biofunctional materials such as a material known to have a skin improving effect. Examples of the material known to have a skin improving effect include proteoglycans, elastin peptides, ceramides, plant extracts, chondroitin sulfates, glucosamines, minerals (e.g., calcium), and vitamins (e.g., vitamin C). In one embodiment, in order to further reduce the bitterness, preferably, the liquid oral composition of the present invention contains a sweetener and an acidulant.

The liquid oral composition of the present invention contains an aqueous medium, usually water. Preferably, the liquid oral composition of the present invention is a liquid oral composition (aqueous liquid oral composition) that uses water as a medium.

The pH of the liquid oral composition of the present invention is preferably 6 or less, more preferably 2 to 6, still more preferably 3 to 4.5, for antiseptic properties. The pH herein is the pH at 25° C.

The pH can be adjusted using an acid or a salt thereof usable in foods and beverages. One type of acids or salts thereof may be used or two or more types of acids or salts thereof may be used in combination.

Preferably, the liquid oral composition of the present invention is used as a beverage (beverage composition). In particular, a soft drink is preferred.

The liquid oral composition of the present invention can be packed in a container. The form of the container is not limited. A sealed container such as a bottle, can, plastic bottle, paper pack, aluminum pouch, or plastic pouch can be used to pack the liquid oral composition so as to provide a packaged beverage (a beverage in a container) or the like.

The method of producing the liquid oral composition of the present invention is not limited. For example, the method preferably includes a mixing step of mixing the components. The liquid oral composition of the present invention can be prepared by mixing components such as Hyp-Gly and a C2-C4 mono- to trihydric alcohol such that the amount of Hyp-Gly is 1.0 mg/100 mL.

In the mixing step, preferably, the components are mixed by adding an aqueous medium. Usually, the aqueous medium is water. The order of mixing the components is not limited as long as the components are mixed uniformly. The production of the liquid oral composition may include a pH adjusting step of adjusting the pH of the liquid oral composition. The pH adjusting step may be performed simultaneously with or after the mixing step. In the production of the liquid oral composition, steps such as filtration, dilution, and sterilization may be suitably performed, if necessary. When the liquid oral composition is provided as a packaged beverage, a step of filling a container with the liquid oral composition may be performed.

<Method of Reducing Bitterness of Liquid Oral Composition>

The present invention also encompasses a method of reducing the bitterness of a liquid oral composition, the method including: adding a C2-C4 mono- to trihydric alcohol to a liquid oral composition containing Hyp-Gly in an amount of 1.0 mg/100 mL or more.

The bitterness of Hyp-Gly in a liquid oral composition containing Hyp-Gly in an amount of 1.0 mg/100 mL or more can be reduced by adding a C2-C4 mono- to trihydric alcohol to the liquid oral composition.

The alcohol may be added by any method at any timing, as long as the liquid oral composition containing Hyp-Gly ultimately contains a C2-C4 mono- to trihydric alcohol. A preferred range of the amount of Hyp-Gly in the liquid oral composition is the same as that in the liquid oral composition of the present invention described above. The alcohol, preferred embodiments thereof, an amount thereof, and the like are the same as those in the liquid oral composition of the present invention described above. The liquid oral composition may contain collagen peptides containing Hyp-Gly. The liquid oral composition may contain other components such as the sweetener described above.

### EXAMPLES

The followings are examples that more specifically describe the present invention. The present invention is not limited to these examples.

### Example 1

A liquid oral composition (beverage) was prepared by mixing Hyp-Gly (OG) (available from SACHEM) and ethanol with water such that the amounts of OG and ethanol were as shown in Table 1. A linear OG was used.

### Examples 2, 3, 4 and 5

Liquid oral compositions were prepared as in Example 1, except that OG and ethanol were added such that the amounts of OG and ethanol were as shown in Table 1.

### Comparative Example 1

A liquid oral composition was prepared as in Example 1, except that ethanol was not added. Specifically, a liquid oral composition was prepared by mixing OG with water such that the amount of OG was as shown in Table 1.

In the liquid oral compositions of the examples, the ratio of the amount of ethanol to the amount of Hyp-Gly (OG) (i.e., the amount of ethanol (vol %)/the amount of Hyp-Gly (mg/100 mL)) was as follows: 0.0007 in Example 1; 0.003 in Example 2; 0.03 in Example 3; 0.01 in Example 4; and 0.015 in Example 5.

The liquid oral compositions obtained in the examples and comparative examples were subjected to sensory evaluation at room temperature by the following method. Table 1 shows the evaluation results.

<Flavor Evaluation>

Three expert panelists performed sensory evaluation of the flavor of each liquid oral composition (at room temperature) in terms of bitterness. The degree of bitterness was sensorily evaluated by each panelist based on the following criteria (with three points being the highest score), and an average score of the three panelists was determined. The results were expressed by average.

Criteria for Flavor Evaluation

3 points: No bitterness was tasted.

2 points: Bitterness was slightly tasted but was not a problem.

1 point: Bitterness was tasted.

TABLE 1

| | | | | | | | Comparative |
|---|---|---|---|---|---|---|---|
| | | Example 1 | Example 2 | Example 3 | Example 4 | Example 5 | Example 1 |
| Liquid oral | OG (mg/100 mL) | 1.5 | 30.0 | 30.0 | 100.0 | 100.0 | 1.5 |
| composition | Ethanol (vol %) | 0.001 | 0.1 | 1.0 | 1.0 | 1.5 | — |
| Evaluation | Bitterness | 1.7 | 3.0 | 1.7 | 1.7 | 1.7 | 1.0 |
| results | | | | | | | |

Example 1 and Comparative Example 1 show that the bitterness of OG (Hyp-Gly) was reduced (suppressed) by adding ethanol. The bitterness of OG was reduced in each of Examples 1 to 4. No alcohol odor was noticed in each of Examples 1 to 4. In Example 5, the bitterness of OG was reduced, but alcohol odor was noticed.

### Example 6 and Example 7

Liquid oral compositions were prepared as in Example 1, except that OG (available from BACHEM) and ethanol were added such that the amounts of OG and ethanol were as shown in Table 2. In these liquid oral compositions, the ratio of the amount of ethanol to the amount of Hyp-Gly (i.e., the amount of ethanol (vol %)/the amount of Hyp-Gly (mg/100 mL)) was as follows: 0.0004 in Example 6 and 0.0003 in Example 7.

### Example 8

Propylene glycol was used instead of ethanol. A liquid oral composition was prepared by mixing OG (available from BACHEM) and propylene glycol with water such that the amounts of OG and propylene glycol were as shown in Table 2. In the liquid oral composition of Example 8, the ratio of the amount of propylene glycol to the amount of Hyp-Gly (i.e., the amount of propylene glycol (vol %)/the amount of Hyp-Gly (mg/100 mL)) was 0.0003.

The liquid oral compositions prepared in Examples 6 to 8 were subjected to flavor evaluation by the method described above. Table 2 shows the results. The bitterness of OG was reduced by adding ethanol or propylene glycol. No alcohol odor was noticed in Examples 6 to 8.

TABLE 2

| | | | Example 6 | Example 7 | Example 8 |
|---|---|---|---|---|---|
| Liquid oral | OG (mg/100 mL) | | 28.0 | 35.0 | 35.0 |
| composition | Alcohol | Ethanol | 0.01 | 0.01 | — |
| | (vol %) | Propylene | — | — | 0.01 |
| | | glycol | | | |

| | | | | |
|---|---|---|---|---|
| Evaluation | Bitterness | 2.3 | 2.3 | 2.3 |
| results | | | | |

### INDUSTRIAL APPLICABILITY

The present invention is useful in the food and beverage field and the like.

1. A liquid oral composition comprising:
Hyp-Gly in an amount of 1.0 mg/100 mL or more; and
a C2-C4 mono- to trihydric alcohol.

2. The liquid oral composition according to claim 1,
wherein an amount of the alcohol is 0.001 to 1.0 vol %.

3. The liquid oral composition according to claim 1,
wherein the alcohol is at least one selected from the group consisting of ethanol, butylene glycol, propylene glycol, and glycerol.

4. The liquid oral composition according to claim 1,
wherein an amount of the Hyp-Gly is 1.0 to 100 mg/100 mL.

5. The liquid oral composition according to claim 1,
wherein the liquid oral composition is a beverage.

6. A method of reducing the bitterness of a liquid oral composition, the method comprising:
adding a C2-C4 mono- to trihydric alcohol to a liquid oral composition containing Hyp-Gly in an amount of 1.0 mg/100 mL or more.

7. The liquid oral composition according to claim 2,
wherein the alcohol is at least one selected from the group consisting of ethanol, butylene glycol, propylene glycol, and glycerol.

8. The liquid oral composition according to claim 2,
wherein an amount of the Hyp-Gly is 1.0 to 100 mg/100 mL.

9. The liquid oral composition according to claim 3,
wherein an amount of the Hyp-Gly is 1.0 to 100 mg/100 mL.

10. The liquid oral composition according to claim 2,
wherein the liquid oral composition is a beverage.

11. The liquid oral composition according to claim 3,
wherein the liquid oral composition is a beverage.

12. The liquid oral composition according to claim 4,
wherein the liquid oral composition is a beverage.
